(19) United States
(12) Patent Application Publication
(10) Pub. No.: US 2024/0082244 A1
(43) Pub. Date: Mar. 14, 2024

(54) PHARMACEUTICAL FORMULATION

(71) Applicant: Redx Pharma PLC, Cheshire (GB)

(72) Inventors: Richard Armer, Cheshire (GB); Marce De Matas, Lancashire (GB)

(21) Appl. No.: 18/271,913

(22) PCT Filed: Jan. 14, 2022
(86) PCT No.: PCT/GB22/50083

§ 371 Date: Jul. 12, 2023

(30) Foreign Application Priority Data
Jan. 15, 2021 (GB) 2100526.9

(51) Int. Cl. A61K 31/497; A61K 9/48

(52) U.S. Cl. CPC A61K 31/497; A61K 9/4825; A61K 9/4833; A61K 9/4866

(57) ABSTRACT

This invention relates to pharmaceutical formulations and a method of making a pharmaceutical formulation. In particular, the invention relates to pharmaceutical formulations and a method of making a pharmaceutical formulation comprising Compound I and a diluent.

This invention relates to a pharmaceutical formulation and a method of making a pharmaceutical formulation. In particular, the invention relates to pharmaceutical formulations and a method of making a pharmaceutical formulation comprising Compound I and a diluent.

The pharmaceutical formulations of the invention may be used in treating conditions mediated by the Wnt signalling pathway such as cancer, e.g. biliary cancer, or enhancing the effectiveness of an anti-cancer treatment.

The described invention provides pharmaceutical formulations proposed to inhibit Wnt-mediated signalling. This includes paracrine signalling in the tissues surrounding tumours and autocrine and paracrine signalling in cancer cells.

### BACKGROUND

The Wnt genes encode a large and highly conserved family of secreted growth factors. During normal development, transcription of Wnt family genes is tightly regulated both temporally and spatially. To date, 19 Wnt proteins have been discovered in humans. All of the Wnt proteins are 38- to 43-kDa cysteine-rich glycoproteins. Wnts have a range of roles during development, governing cell fate, migration, proliferation and death. In adults the role of Wnts is thought to be linked to maintaining tissue homeostasis with aberrant signalling implicated in a variety of cancers.

Wnt proteins undergo post-translational modification, shown in several mutation experiments to be vital for effective protein trafficking and secretion (Tang, et al. (2012) Dev. Biol 364, 32-41, Takada, R. et al (2006) Dev. Cell 11, 791-801). Palmitoylation of Wnt proteins occurs at several conserved amino acids (C77, S209) and is performed by porcupine, an O-acetyltransferase, in the endoplasmic reticulum. Mutations in porcupine have been shown to be the cause of developmental disorders, including focal dermal hypoplasia, through impaired Wnt pathway signalling (Grzeschik, et al. (2007) Nat. Genet, 39 pp. 833-835). The dependence of Wnt ligand signalling on porcupine and the body of evidence linking Wnt pathway signalling to cancer has led to porcupine being identified as a potential anti-cancer target.

WO 2016/055786 relates to novel small molecule inhibitors of porcupine (PORCN) which prevents the secretion of wingless-type MMTV Integration Site (Wnt) ligands and downstream activation of the pathway. The compounds disclosed have been shown to have activity in relevant ring finger protein 43 (RNF43) mutant cancer models both in vitro and in vivo and a therapeutic margin has been demonstrated in vivo. RNF43 mutations are known to occur in both gastric cancer and pancreatic cancer. Further, preclinical evidence suggests that biliary cancer patients will be sensitive to PORCN inhibition due to high Wnt ligand drive.

This invention relates to formulations of a particular compound from WO2016/055786, referred to herein as Compound I.

Previously pharmaceutical formulations of Compound I have been prepared as a solid blend formulation containing drug substance at strengths of 5.0 mg, 10.0 mg, 20.0 mg and 40.0 mg, with the drug substance blended with conventional excipients for such formulations. However, it has since been found that lower dosage strengths are required. In particular, per capsule dosages of 0.5 mg and 1.0 mg are now required. It is difficult to achieve the required homogeneity and content uniformity of capsules using conventional manufacturing approaches at lower dosage strengths in a solid blend formulation.

It is an aim of certain embodiments of this invention to provide a pharmaceutical formulation comprising Compound I in an effective dose. It is an aim of certain embodiments of this invention to provide a pharmaceutical formulation comprising Compound I in a low dose, e.g. a lower strength than is described in certain clinical studies.

It is an aim of certain embodiments of this invention to provide method of making a pharmaceutical formulation comprising Compound I.

It is an aim of certain embodiments of this invention to provide method of making a pharmaceutical formulation comprising Compound I which is homogeneous at low doses. It is an aim of certain embodiments of this invention to provide method of making a pharmaceutical formulation comprising Compound I which is homogeneous at low doses within a batch, e.g. which complies with requirements for uniformity of dosage units (content uniformity) described in relevant pharmacopoeia, an example being section 2.9.40 of the European Pharmacopoeia. It is an aim of certain embodiments of this invention to provide method of making a pharmaceutical formulation comprising Compound I which is of consistent strength in relation to Compound 1 at low doses between capsules with both the mean and individual dosage units demonstrating active agent content in the range 90-110% of the label claim as measured by HPLC analysis.

It is an aim of certain embodiments of this invention to provide a method of making a stable pharmaceutical formulation comprising Compound I which is homogeneous at low doses.

Certain embodiments of this invention satisfy some or all of the above aims.

### BRIEF SUMMARY OF THE DISCLOSURE

In accordance with a first aspect of the present invention there is provided a semi-solid pharmaceutical formulation comprising:

### Compound I;

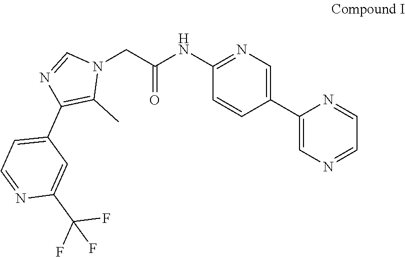 [SMILES: Cc1c(-c2ccnc(C(F)(F)F)c2)ncn1CC(=O)Nc1ccc(-c2cnccn2)cn1]

distributed in a semi-solid diluent.

By combining Compound I with a semi-solid diluent, the inventors discovered that it was possible to achieve the desired homogeneity of the API in the formulation. The resulting formulation can be encapsulated in a capsule. For example, a gelatin capsule may be used. The capsule can then be administered to a patient in need thereof.

The semi-solid diluent may be an amphiphilic substance in which a polyalkylene glycol, e.g. a polyethylene glycol, is conjugated to a lipophilic or hydrophobic molecular entity. The polyethylene glycol chain may comprise from 3 to 70 alkylene glycol (e.g. ethylene glycol) units. Semi-solid formulations formed with these diluents are particularly beneficial in achieving a stable formulation.

The melting point of the semi-solid diluent may range from 40° C. to 55° C. e.g. from 42° C. to 51° C. The theoretical HLB ((Hydrophilic Lipophilic balance, i.e. the degree to which the component is hydrophilic or lipophilic) of the semi-solid diluent may range from 10 to 20 e.g. from 12 to 17.

In some embodiments, the semi-solid formulation comprises a Gelucire® component. Following the name Gelucire®, there is a set of two figures: the first (e.g. 44) indicates the melting point in degrees Celsius, the second (e.g. 14) indicates the theoretical HLB (Hydrophilic Lipophilic balance) i.e. the degree to which the component is hydrophilic or lipophilic. The melting point of the Gelucire® component may range from 40° C. to 55° C. e.g. e.g. from 42° C. to 51° C., e.g. 44° C., 48° C. or 50° C. The theoretical HLB may range from 1 to 20, e.g. from 12 to 17, e.g. 01, 13, 14 or 16.

In some embodiments, the semi-solid diluent is selected from the group comprising polyethylene glycol e.g. PEG (e.g. PEG1000 or higher Mw variants), a water soluble surfactant e.g. Gelucire® 48/16, a water dispersible surfactant e.g. Gelucire® 44/14 or Gelucire® 50/13, or a hard fat e.g. Gelucire® 43/01, Vitamin E polyethylene glycol succinate (TPGS), polyoxyl 35 castor oil e.g. Kolliphor EL, polyoxyl 40 castor oil e.g. Kolliphor RH40, Solutol HS15 (polyoxyethylene esters of 12-monohydroxy stearic acid), glyceryl stearate (Imwitor 900), glyceryl caprate/caprylate ester such as Capmul MCM. In some embodiments, the semi-solid diluent is selected from the group comprising a water soluble surfactant, a water dispersible surfactant, a hard fat, Vitamin E polyethylene glycol succinate, medium chain triglycerides, polyoxyl 35 castor oil.

In some embodiments, the semi-solid diluent is selected from the group comprising Vitamin E polyethylene glycol succinate and lauroyl macrogol-32 glycerides. Thus, in some embodiments, the semi-solid diluent is Vitamin E polyethylene glycol succinate. Thus, in some embodiments, the semi-solid diluent is a pegylated lipid, for example Gelucire® 44/14 (lauroyl macrogol-32 glycerides). In some embodiments, the semi-solid diluent is Gelucire® 48/16, (macrogol-32 stearate) Gelucire® 50/13 (stearoyl macrogol-32 glycerides) or Gelucire® 43/01 (hard fat). Gelucire® 48/16 is a polyethylene glycol monostearate (type I) NF and consists of PEG-32 (MW 1500) esters of palmitic (C16) and stearic (C18) acids. It is a solid at ambient temperature. Gelucire® 44/14 is a lauroyl polyoxyl/macrogol 32 glycerides NF/EP and consists of a small fraction of mono, di- and triglycerides and mainly PEG-32 (MW 1500) mono- and diesters of lauric acid (C12). Gelucire® 50/13 is a stearoyl polyoxyl/macrogol 32 glycerides NF/EP and consists of mono, di- and triglycerides and PEG-32 (MW 1500) mono- and diesters of palmitic (C16) and stearic (C18) acids. Gelucire® 43/01 is composed of mono-, di- and triglyceride esters of fatty acids (C8 to C18), the triester fraction being predominant.

Compound I is particularly compatible with Vitamin E polyethylene glycol succinate and with Gelucire® 44/14, a polyethylene glycol glyceride.

In some embodiments, the semi-solid pharmaceutical formulation further comprises an anti-oxidant. In some embodiments, the anti-oxidant may be selected from butylated hydroxyanisole (BHA) and butylated hydroxytoluene (BHT).

In some embodiments, the semi-solid pharmaceutical formulation further comprises a pharmaceutically acceptable excipient selected from the group comprising sodium lauryl sulphate, poloxamer and other surfactants. In some embodiments, the semi-solid pharmaceutical formulation further comprises a polymer agent, e.g a polymer agent which can inhibit precipitation of Compound I. In some embodiments, the polymer agent is selected from the group comprising polyvinylpyrrolidone (PVP), hydroxypropyl methylcellulose (HPMC), methylcellulose and copovidone.

In some embodiments, the semi-solid pharmaceutical formulation comprises Compound I in an amount of from 0.1% w/w to about 10% w/w. In some embodiments, the semi-solid pharmaceutical formulation comprises Compound I in an amount of from 0.5% w/w to about 5% w/w. In some embodiments, the semi-solid pharmaceutical formulation comprises Compound I in an amount of about 0.5% w/w, about 1% w/w, about 1.5% w/w, about 2% w/w, about 2.5% w/w, about 3% w/w, about 3.5% w/w, about 4% w/w, about 4.5% w/w or about 5% w/w. In some embodiments, the semi-solid pharmaceutical formulation comprises Compound I in an amount of from 0.5% w/w to about 3% w/w. In some embodiments, the semi-solid pharmaceutical formulation comprises Compound I in an amount of from 1.5% w/w to about 2.5% w/w. In some embodiments, the semi-solid pharmaceutical formulation comprises Compound I in an amount of from 1% w/w to about 2% w/w.

In some embodiments, the semi-solid pharmaceutical formulation comprises Compound I in a total dose of from about 0.05 mg to about 5 mg. In some embodiments, the semi-solid pharmaceutical formulation comprises Compound I in a total dose of from about 0.25 mg to about 2.5 mg. In some embodiments, the semi-solid pharmaceutical formulation comprises Compound I in a total dose of from about 0.25 mg to about 1.5 mg. In some embodiments, the semi-solid pharmaceutical formulation comprises Compound I in a total dose of about 0.25 mg, about 0.5 mg, about 0.75 mg, about 1.0 mg, about 1.25 mg, about 1.5 mg, about 1.75 mg, about 2.0 mg, about 2.25 mg or about 2.5 mg. In some embodiments, the semi-solid pharmaceutical formulation comprises Compound I in a total dose of from about 0.5 mg to about 1 mg.

In an embodiment, the semi-solid pharmaceutical formulation comprises: Compound I present in an amount of from 0.5% w/w to 3% w/w; and Vitamin E polyethylene glycol succinate in an amount of from 97% w/w to 99.5% w/w. In an embodiment, the semi-solid pharmaceutical formulation comprises: Compound I present in an amount of from 1% w/w to 2% w/w; and Vitamin E polyethylene glycol succinate in an amount of from 98% w/w to 99% w/w.

In some embodiments, the semi-solid pharmaceutical formulation is encapsulated in a capsule. In some embodiments, the capsule is a gelatin capsule. In some embodiments, the capsule is a vegetable-derived capsule e.g. HPMC. In some embodiments, the semi-solid pharmaceutical formulation is encapsulated in a gelatin capsule, such as a white opaque gelatin capsule (gelatin and titanium dioxide).

In an embodiment, the semi-solid pharmaceutical formulation comprises a total dose of between about 0.5 mg and 2 mg of Compound I and between about 48.0 mg and about 49.5 mg of vitamin E polyethylene glycol succinate. In an embodiment, the semi-solid pharmaceutical formulation comprises a total dose of between about 0.5 mg and 2 mg of Compound I and between about 48 mg and about 49.5 mg of vitamin E polyethylene glycol succinate encapsulated in a gelatin capsule.

In an embodiment, the semi-solid pharmaceutical formulation comprises a total dose of between about 0.75 mg and 1.25 mg of Compound I and between about 48.75 mg and about 49.25 mg of vitamin E polyethylene glycol succinate. In an embodiment, the semi-solid pharmaceutical formulation comprises a total dose of between about 0.75 mg and 1.25 mg of Compound I and between about 48.75 mg and about 49.25 mg of vitamin E polyethylene glycol succinate encapsulated in a gelatin capsule.

In an embodiment, the semi-solid pharmaceutical formulation comprises a total dose of between about 0.25 mg and about 0.75 mg of Compound I and between about 49.25 mg and about 49.75 mg of vitamin E polyethylene glycol succinate. In an embodiment, the semi-solid pharmaceutical formulation comprises a total dose of between about 0.25 mg and about 0.75 mg of Compound I and between about 49.25 mg and about 49.75 mg of vitamin E polyethylene glycol succinate encapsulated in a gelatin capsule.

In an embodiment, the semi-solid pharmaceutical formulation comprises a total dose of about 1 mg of Compound I and about 49 mg of vitamin E polyethylene glycol succinate. In an embodiment, the semi-solid pharmaceutical formulation comprises a total dose of about 1 mg of Compound I and about 49 mg of vitamin E polyethylene glycol succinate encapsulated in a gelatin capsule.

In an embodiment, the semi-solid pharmaceutical formulation comprises a total dose of about 0.5 mg of Compound I and about 49.5 mg of vitamin E polyethylene glycol succinate. In an embodiment, the semi-solid pharmaceutical formulation comprises a total dose of about 0.5 mg of Compound I and about 49.5 mg of vitamin E polyethylene glycol succinate encapsulated in a gelatin capsule.

The semi-solid pharmaceutical formulation of the invention may be for medical use.

The semi-solid pharmaceutical formulation of the invention may be for use in the treatment of cancer.

The invention also provides a method of treating cancer, the method comprising administering to a subject in need thereof a therapeutically acceptable amount of the semi-solid formulation of the first aspect.

The cancer may be selected from sarcoma, melanoma, skin cancer, haematological tumors, lymphoma, carcinoma, adenoma and leukemia. Specific cancer, sarcoma, melanoma, skin cancer, haematological tumors, lymphoma, carcinoma, and leukemia that may be treated by the compound of the invention may be selected from: esophageal squamous cell carcinoma, gastric cancer, glioblastomas, astrocytomas; retinoblastoma, osteosarcoma, chondosarcoma, Ewing's sarcoma, rabdomysarcoma, Wilm's tumor, basal cell carcinoma, non-small cell lung cancer, brain tumour, hormone refractory prostate cancer, prostate cancer, metastatic breast cancer, breast cancer, metastatic pancreatic cancer, pancreatic cancer, colorectal cancer, biliary cancer e.g. cholangiocarcinoma, thymus cancer, cervical cancer, head and neck squamous cell carcinoma and cancer of the head and neck.

The cancer may be a solid tumour. The cancer may be selected from gastric cancer, pancreatic cancer, colorectal cancer, thymus cancer, and biliary cancer e.g cholangiocarcinoma. The cancer may be selected from colorectal cancer, thymus cancer and biliary cancer e.g cholangiocarcinoma. The cancer may be selected from colorectal cancer, pancreatic cancer and biliary cancer e.g cholangiocarcinoma. The cancer may be biliary tract cancer, e,g, cholangiocarcinoma. The cancer may be thymus cancer. The cancer may be colorectal cancer. The cancer may be pancreatic cancer.

The semi-solid pharmaceutical formulation of the invention may be for treatment of a condition which is modulated by Porcn.

The invention also provides a method of treating a condition which is modulated by Porcn, the method comprising administering to a subject in need thereof a therapeutically acceptable amount of the semi-solid formulation of the first aspect.

The condition which is modulated by Porcn may be cancer.

The condition which is modulated by Porcn may be selected from: skin fibrosis, idiopathic pulmonary fibrosis, renal interstitial fibrosis, liver fibrosis, proteinuria, kidney graft rejection, osteoarthritis, Parkinsons's disease, cystoid macular edema, uveitis associated cystoid macular edema, retinopathy, diabetic retinopathy and retinopathy of prematurity.

The semi-solid pharmaceutical formulation of the invention may be for use in the treatment of a condition selected from: skin fibrosis, idiopathic pulmonary fibrosis, renal interstitial fibrosis, liver fibrosis, proteinuria, kidney graft rejection, osteoarthritis, Parkinsons's disease, cystoid macular edema, uveitis associated cystoid macular edema, retinopathy, diabetic retinopathy and retinopathy of prematurity.

The invention also provides a method of treating a condition selected from: skin fibrosis, idiopathic pulmonary fibrosis, renal interstitial fibrosis, liver fibrosis, proteinuria, kidney graft rejection, osteoarthritis, Parkinsons's disease, cystoid macular edema, uveitis associated cystoid macular edema, retinopathy, diabetic retinopathy and retinopathy of prematurity, the method comprising administering to a subject in need thereof a therapeutically acceptable amount of the semi-solid formulation of the first aspect.

In accordance with a second aspect of the present invention there is provided a method of making a semi-solid pharmaceutical formulation, the method comprising the steps of:
(i) melting a semi-solid diluent;
(ii) distributing Compound I in the molten semi-solid diluent to form a molten blend;
(iii) loading the molten blend into a capsule shell.

In some embodiments, the method of the second aspect may be used to provide the semi-solid pharmaceutical formulation of the first aspect and related embodiments.

In some embodiments, step (ii) comprises dissolving Compound I in the semi-solid diluent. In some embodiments, step (ii) further comprises mixing Compound I with the melted semi-solid diluent of step (i).

In some embodiments, the method may further comprise the step of passing Compound I through a screen to remove any lumps prior to the step of dissolving Compound I in the diluent.

In some embodiments, Compound I and the molten diluent are continuously mixed together using a suitable mixer to produce a uniform solution.

Capsule filling may be undertaken using conventional capsule filling methods and equipment suitable for use with molten semisolid formulations. Capsules can be filled using a range of methods in which a fixed volume of formulation is dispensed into an empty capsule shell. This may include methods of hand filling using positive displacement pipettes or using automated capsule filling machines. In an illustrative filling process, the body of the capsule and its cap are separated, with the body immobilised using a capsule holder. When filling by hand, a pipette is used to measure and dispense a known volume and weight of the formulation into the capsule body. Once filling is complete, the cap is returned and fixed onto the capsule body by hand. When filling using automated systems, commercially available machines are used, which achieve the same functions as filling by hand.

In some embodiments, the step of melting the semi-solid diluent is performed at a temperature of between about 30° C. and about 90° C. In some embodiments, the step of melting the semi-solid diluent is performed at a temperature of between about 40° C. and about 80° C. In some embodiments, the step of melting the semi-solid diluent is performed at a temperature of about 40° C., 45° C., 50° C., 55° C., 60° C., 65° C., 70° C., 75° C. or about 80° C. In some embodiments, the step of melting the semi-solid diluent is performed at a temperature of between about 45° C. and about 80° C. In some embodiments, the step of melting the semi-solid diluent is performed at a temperature of about 50° C. to about 60° C. In some embodiments, the step of melting the semi-solid diluent is performed at a temperature of about 55° C.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “semi-solid” refers to a substance having both liquid and solid properties at room temperature i.e. having a viscosity and rigidity intermediate between that of a solid and a liquid. At lower temperatures, the substance may display solid properties. At higher temperatures, the substance may display liquid properties. The term “semi-solid” may refer to a waxy solid which is a wax at room temperature but which softens and/or melts at elevated temperatures.

The term “stable” refers to both chemical and physical stability. The term “stable” may refer to chemical and/or physical stability of a compound or composition over a period of time (e.g. 6 months) at about 20° C. to about 40° C. and about 50% RH to about 80% RH. For example, the compound or composition may be chemically and/or physically stable over a period of 24 hours. The compound or composition may be chemically and/or physically stable over a period of up to 6 months. The compound or composition may be chemically and/or physically stable over a period of up to 12 months. The compound or composition may be chemically and/or physically stable over a period of over 12 months. The compound or composition may be chemically and/or physically stable over a period of up to 24 or up to 36 months. It may be that the composition is physically and chemically stable for 6 months at 40° C./75% RH. It may be that the composition is physically and chemically stable for 24 months at 25° C./60% RH.

The reader's attention is directed to all papers and documents which are filed concurrently with or previous to this specification in connection with this application and which are open to public inspection with this specification, and the contents of all such papers and documents are incorporated herein by reference.

### EXAMPLES

### Example 1

Compound I can be made according to the procedures outlined in the General Schemes of WO 2016/055786. Alternatively, Compound I can be made according to the procedure outlined in Example 9 of WO 2016/055786.

An experimental stability/excipient compatibility study was performed and demonstrated that Compound I is compatible with Vitamin E polyethylene glycol succinate and the gelatin capsule shell, in a prototype formulation at 25° C./60% RH and 40° C./75% RH over a 1-month period. No significant degradation of Compound I was observed and therefore the combination of Compound I, Vitamin E polyethylene glycol succinate and the gelatin capsule shell was selected for use in capsules comprising Compound I. A stability evaluation of a selected formulation to support establishment of shelf-life has been reported in Example 2. Small scale manufacturability studies also showed that the formulation could be filled consistently to the required fill weights into gelatin capsules to deliver acceptable assay and uniformity of dosage units (content uniformity). A homogeneous solution of Compound I is formed with the Vitamin E polyethylene glycol succinate (TPGS), which enables good uniformity of content to be achieved at low strengths.

### Example 2

0.5 mg and 1 mg capsules of Compound I are manufactured from a blend of drug substance at a level of 1% w/w and 2% w/w respectively, which is mixed with excipient. The batch formulae for a batch size of 500 g of Compound I Capsules Blend is shown in Table 1 for the two different capsule strengths. The Compound I Capsules Blend batch size may be adjusted as necessary to support the needs of the clinical programme with components adjusted on a pro-rata basis.

TABLE 1
Batch Formulae for Compound I Capsules Blend

| | Quantity per | Quantity per |
|---|---|---|
| | 500 g batch | 500 g batch |
| | (g) - 0.5 mg | (g) - 1.0 mg |
| Component | capsules | capsules |
| Compound I | 5.00* | 10.00* |
| Vitamin E polyethylene glycol succinate | 495.00 | 490.00 |
| Total per batch | 500.00 | 500.00 |

*The amount of Compound I included in the formulation may be corrected for purity, water and solvents.
The Vitamin E polyethylene glycol succinate level will also be adjusted accordingly to maintain the same total blend weight.

Description of the Process

Compound I capsules are manufactured from a blend of drug substance and excipient according to the batch formulae described in Table 1 using conventional mixing and capsule filling processes according to Good Manufacturing Practice. Vitamin E polyethylene glycol succinate is melted with a product temperature not less than 45° C. but not exceeding 80° C. Compound I is passed through an appropriately sized screen (pre-screen) if necessary, to remove any lumps and is then added to the molten Vitamin E polyethylene glycol succinate. Both components are continuously mixed together using a suitable mixer to produce a uniform solution as determined visually. The appropriate volume of the molten blend is accurately transferred into the gelatin capsule shells to achieve the target capsule fill weight (see Table 2). In this particular example, filling has been conducted by hand, in which the capsule body and cap are manually separated, and the body immobilised using a capsule holder. A pipette is used to measure and dispense a known volume and weight of the molten formulation into the capsule, following which, the cap and body are joined together by hand. The molten blend is continuously mixed during the capsule filing process at a temperature suitable to maintain the formulation in the molten state as stated above.

TABLE 2
Composition of Compound I Drug Product

| | | | Quantity/Capsule | | | |
|---|---|---|---|---|---|---|
| | Quality | | 0.5 mg | | 1.0 mg | |
| Component | Standard | Function | Mg | % w/w | mg | % w/w |
| Compound I g | Section | Active | 0.5 | 1.0 | 1.0 | 2.0 |
| Substance | 2.1.S.4.1 | Ingredient | | | | |
| Vitamin E | USP/NF | Diluent | 49.5 | 99.0 | 49.0 | 98.0 |
| polyethylene | | | | | | |
| glycol succinate | | | | | | |
| (TPGS) | | | | | | |
| White opaque | Ph. Eur for | Encapsulation | Size #3 | — | Size #3 | — |
| gelatin capsules | gelatin and | | | | | |
| | titanium | | | | | |
| | dioxide | | | | | |
| Total | — | — | 50 | 100 | 50 | 100 |

Manufacturing Process for Compound I and Controls

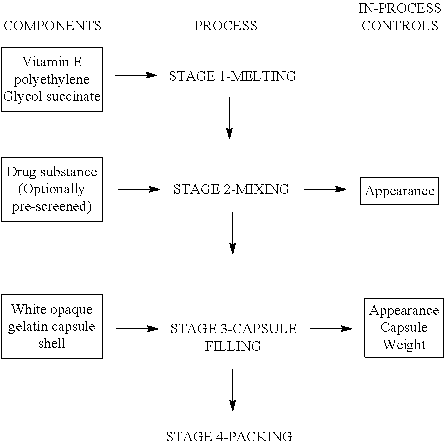

Samples are tested in accordance with the following method: A summary of analytical procedures used in testing of Compound I in 0.5 mg and 1.0 mg capsules is given below.

a) Appearance (AM003) Visual inspection of Compound I capsules should reveal a size #3 white opaque capsule free from physical defects. b) Identity, Content Uniformity, Assay and Related Substances by UPLC (AM402) A UHPLC reversed phase method is used for the assay/related substances, content uniformity and identification testing of the Compound I capsules and is suitable for testing 0.5 mg and 1.0 mg capsules. The chromatographic conditions are detailed in Table 3:

TABLE 3
UHPLC Retention time for Compound:

| Parameter | Value | | | |
|---|---|---|---|---|
| Method: | UPLC with PDA detection at 295 nm | | | |
| Column: | Phenomenex Kinetex C18, 100 A, 100 × 4.6 | | | |
| | mm, 2.6 μm | | | |
| Column temperature: | 35° C. | | | |
| Flow rate: | 1.0 mL/min | | | |
| Injection volume: | 3 μL + auto dilution (3 μL) | | | |
| Mobile Phase A | 20 mM Sodium Acetate Buffer pH 4.5 | | | |
| Mobile Phase B | Acetonitrile | | | |
| Mobile Phase C | 2-Propanol | | | |
| Gradient Conditions | Time | % A | % B | % C |
| | 0.0 | 95 | 5 | 0 |
| | 5.0 | 95 | 5 | 0 |
| | 8.0 | 65 | 35 | 0 |
| | 15.0 | 40 | 60 | 0 |
| | 15.1 | 5 | 45 | 50 |
| | 20.0 | 5 | 45 | 50 |
| | 20.1 | 95 | 5 | 0 |
| | 25.0 | 95 | 5 | 0 |

| | |
|---|---|
| Approx. Retention Time | 10.5 minutes |

A working standard (System Suitability Standard) is prepared at 0.1 mg/mL in diluent, in duplicate for assay and at 0.02 mg/mL in diluent for content uniformity. Sensitivity standard solutions are prepared at 0.1% and 0.05% by dilution of the working standard solution. Content Uniformity Sample Preparation: Working sample solutions for all dose strengths are prepared at 0.02 mg/mL Assay/Related Substance Sample Preparation: Working sample solutions for all dose strengths are prepared at 0.1 mg/mL, in duplicate.

The batch analysis data for the Compound I 0.5 mg and 1.0 mg capsules are given in Table 4.

TABLE 4
Batch analysis data for the Compound I 0.5 mg and 1.0 mg capsules

| | | Results | | | | |
|---|---|---|---|---|---|---|
| Test | Specification | 0.5 mg Batch | 1.0 mg Batch B | 0.5 mg Batch | 1.0 mg Batch | 1.0 mg Batch |
| Assay (by | 90-110% of | 99% | 101% | 97% | 102% | 102% |
| HPLC) | the label claim | | | | | |
| Related | Unspecified | 0.06% (RRT0.42) | 0.06% (RRT0.42) | 0.2% (RRT0.42) | 0.2% (RRT0.42) | 0.2% (RRT0.42) |
| substances | impurities and | 0.05% (RRT1.20) | 0.05% (RRT1.20) | 0.2% (Total) | 0.2% (Total) | 0.2% (Total) |
| (by HPLC) | degradation | 0.11%(Total) | 0.12%(Total) | | | |
| | products: | | | | | |
| | NMT 1.0% | | | | | |
| | area | | | | | |
| | Total | | | | | |
| | impurities and | | | | | |
| | degradation | | | | | |
| | products NMT | | | | | |
| | 3.0% area | | | | | |
| Uniformity | Complies with | Complies | Complies | Complies | Complies | Complies |
| of dosage | Ph Eur 2.9.40 | | | | | |
| units | | | | | | |
| (Content | | | | | | |
| Uniformity) | | | | | | |

The drug products described in Table 2 have been shown to be physically and chemically stable for 6 months at 40° C./75% RH and for 24 months at 25° C./60% RH.

The drug products described in Table 2 have been administered to patients in a Phase I clinical study in adults with advanced solid tumours (e.g. colorectal cancer, biliary tract cancer, thymus cancer). Pharmacokinetic studies showed that the drug products described in Table 2 facilitate good absorption and relevant exposure of the drug. Furthermore, preliminary clinical efficacy was observed in some patients with potential Wnt ligand dependent biliary, colorectal and thymus tumours.

### Example 3

Four batches of Compound I, two each of 0.5 mg and 1.0 mg capsules have been set down on accelerated, intermediate and long-term storage evaluation to support shelf-life determination to allow for further extension of shelf-life.

TABLE 5
Shelf-life determination

| | | Date of | | |
|---|---|---|---|---|
| | | Manufacture | | Stability |
| | | (stability | Storage | timepoints |
| Strength | Use | start date) | Conditions | (months) |
| 0.5 mg | Development | 15 Oct. 2018 | 25° C./ | 0, 1, 3, 6, |
| | | | 60% RH | 12, 24, 36 |
| | | (26 Oct. 2018) | 30° C./ | 0, 1, 3, 6, |
| | | | 65% RH | 12 |
| | | | 40° C./ | 0, 1, 3, 6 |
| | | | 75% RH | |
| 1.0 mg | Development | 16 Oct. 2018 | 25° C./ | 0, 1, 3, 6, |
| | | | 60% RH | 12, 24, 36 |
| | | (26 Oct. 2018) | 30° C./ | 0, 1, 3, 6, |
| | | | 65% RH | 12 |
| | | | 40° C./ | 0, 1, 3, 6 |
| | | | 75% RH | |
| 0.5 mg | Clinical | 30 Nov. 2018 | 25° C./ | 0, 1, 2, 3, 6, |
| | | | 60% RH | 9, 12, 18, 24, 36 |
| | | (22 Jan. 2019) | 30° C./ | 0, 1, 2, 3, 6, |
| | | | 65% RH | 9, 12, 18, 24, 36 |
| | | | 40° C./ | 0, 1, 2, 3, 6 |
| | | | 75% RH | |
| 1.0 mg | Clinical | 4 Dec. 2018 | 25° C./ | 0, 1, 2, 3, 6, |
| | | | 60% RH | 9, 12, 18, 24, 36 |
| | | (22 Jan. 2019) | 30° C./ | 0, 1, 2, 3, 6, |
| | | | 65% RH | 9, 12, 18, 24, 36 |
| | | | 40° C./ | 0, 1, 2, 3, 6 |
| | | | 75% RH | |

Samples were tested in accordance with the methods in Example 2. Samples are tested for assay and related substances as per the specification in Table 6.

TABLE 6
Shelf-life specification for Compound I capsules

| Test | Acceptance criteria |
|---|---|
| Assay | 90.0-110.0% of label claim |
| Related substances | |
| Reporting threshold | 0.1% |
| Unspecified impurity & degradation product | NMT 1.0% |
| Total impurities & degradation products | NMT 3.0% |

TABLE 7
Stability Data for 0.5 mg Compound I Capsules (Accelerated 40° C./75% RH)
Compound I 0.5 mg capsules; Accelerated 40° C./75% RH

| | Acceptance | | | | |
|---|---|---|---|---|---|
| Test | criteria | T0 | T1 | T3 | T6 |
| Assay | 90.0-110.0% | 99% | 101% | 99% | 97% |
| | of label claim | | | | |
| Related substances | | | 0.17% (RRT 0.41) | | 0.51% (RRT 0.42) |
| Reporting threshold | 0.1% | 0.06% (RRT 0.42) | 0.07% (RRT 1.04) | 0.28% (RRT 0.42) | 0.08%(RRT0.74) |
| Unspecified impurity & | NMT 1.0% | 0.05% (RRT 1.20) | 0.06% (RRT 1.20) | 0.05% (RRT 1.20) | 0.06% (RRT 1.20) |
| deg. product | | | | | |
| Total impurities & | NMT 3.0% | 0.11% (Total) | 0.30% (Total) | 0.33% (Total) | 0.64% (Total) |
| deg. Products | | | | | |

TABLE 8
Stability Data for 0.5 mg Compound I Capsules (Intermediate 30° C./65% RH)
Compound I 0.5 mg capsules; Intermediate 30° C./65% RH

| | Acceptance | | | | | |
|---|---|---|---|---|---|---|
| Test | criteria | T0 | T1 | T3 | T6 | T12 |
| Assay | 90.0-110.0% | 99% | 101% | 101% | 99% | 100% |
| | of label claim | | | | | |
| Related substances | | | | | | |
| Reporting threshold | 0.1% | 0.06% (RRT 0.42) | 0.12% (RRT 0.40) | 0.15% (RRT 0.42) | 0.16% (RRT 0.42) | 0.36% (RRT 0.43) |
| Unspecified impurity & | NMT 1.0% | 0.05% (RRT 1.20) | 0.05% (RRT 1.20) | 0.06% (RRT 1.20) | 0.05% (RRT 1.20) | 0.05% (RRT 1.19) |
| deg. product | | | | | | |
| Total impurities & | NMT 3.0% | 0.11% (Total) | 0.17% (Total) | 0.21% (Total) | 0.21% (Total) | 0.41% (Total) |
| deg. Products | | | | | | |

TABLE 9
Stability data for 0.5 mg Compound I Capsules (Long term 25° C./60% RH)
Compound I 0.5 mg capsules; Long term 25° C./60% RH

| | Acceptance | | | | | |
|---|---|---|---|---|---|---|
| Test | criteria | T0 | T1 | T3 | T6 | T12 |
| Assay | 90.0-110.0% of | 99% | 100% | 99% | 99% | 100% |
| | label claim | | | | | |
| Related substances | | | | | | |
| Reporting threshold | 0.1% | 0.06% (RRT 0.42) | 0.10% (RRT 0.40) | 0.10% (RRT 0.42) | 0.12% (RRT 0.42) | 0.29% (RRT 0.42) |
| Unspecified impurity & | NMT 1.0% | 0.05% (RRT 1.20) | 0.05% (RRT 1.20) | 0.06% (RRT 1.20) | 0.05% (RRT 1.20) | 0.05% (RRT 1.19) |
| deg. product | | | | | | |
| Total impurities & | NMT 3.0% | 0.11% (Total) | 0.16% (Total) | 0.16% (Total) | 0.17% (Total) | 0.34% (Total) |
| deg. Products | | | | | | |

TABLE 10
Stability data for 1.0 mg Compound I Capsules (Accelerated 40° C./75% RH)
Compound I 1.0 mg capsules; Accelerated 40° C./75% RH

| | Acceptance | | | | |
|---|---|---|---|---|---|
| Test | criteria | T0 | T1 | T3 | T6 |
| Assay | 90.0-110.0% | 101% | 103% | 99% | 101% |
| | of label claim | | | | |
| Related substances | | | | | |
| Reporting threshold | 0.1% | 0.06% (RRT 0.42) | 0.16% (RRT 0.41) | 0.29% (RRT 0.42) | 0.46% (RRT 0.42) |
| Unspecified impurity | NMT 1.0% | 0.05% (RRT 1.20) | 0.05% (RRT 1.20) | 0.05% (RRT 1.20) | 0.46% (Total) |
| & deg. product | | | | | |
| Total impurities & | NMT 3.0% | 0.12% (Total) | 0.21% (Total) | 0.35% (Total) | |
| deg. Products | | | | | |

TABLE 11
Stability data for 1.0 mg Compound I Capsules (Intermediate 30° C./65% RH)
Compound I 1.0 mg capsules; Intermediate 30° C./65% RH

| | Acceptance | | | | | |
|---|---|---|---|---|---|---|
| Test | criteria | T0 | T1 | T3 | T6 | T12 |
| Assay | 90.0-110.0% | 101% | 102% | 103% | 103% | 101% |
| | of label claim | | | | | |
| Related substances | | | | | | |
| Reporting threshold | 0.1% | 0.06% (RRT0.42) | 0.11% (RRT 0.41) | 0.11% (RRT 0.42) | 0.19% (RRT 0.42) | 0.26% (RRT 0.43) |
| Unspecified impurity & | NMT 1.0% | 0.05% (RRT 1.20) | 0.05% (RRT 1.20) | 0.05% (RRT 1.20) | 0.05% (RRT 1.20) | 0.05% (RRT 1.19) |
| deg. product | | | | | | |
| Total impurities & | NMT 3.0% | 0.12% (Total) | 0.16% (Total) | 0.17% (Total) | 0.24% (Total) | 0.31% (Total) |
| deg. Products | | | | | | |

TABLE 12
Stability Data for 1.0 mg Compound I Capsules (Long-term 25° C./60% RH)
Compound I 1.0 mg capsules; Long-term 25° C./60% RH

| | Acceptance | | | | | |
|---|---|---|---|---|---|---|
| Test | criteria | T0 | T1 | T3 | T6 | T12 |
| Assay | 90.0-110.0% | 101% | 102% | 103% | 101% | 101% |
| | of label claim | | | | | |
| Related substances | | | | | | |
| Reporting threshold | 0.1% | 0.06% (RRT0.42) | 0.10% (RRT0.40) | 0.09% (RRT0.42) | 0.14% (RRT0.42) | 0.38% (RRT0.43) |
| Unspecified impurity & | NMT 1.0% | 0.05% (RRT1.20) | 0.06% (RRT1.20) | 0.05% (RRT1.20) | 0.05% (RRT1.20) | 0.05% (RRT1.19) |
| deg. product | | | | | | |
| Total impurities & | NMT 3.0% | 0.12%(Total) | 0.16% (Total) | 0.15% (Total) | 0.20% (Total) | 0.43% (Total) |
| deg. Products | | | | | | |

TABLE 13
Stability data for 0.5 mg Compound I GMP Clinical Capsules (Accelerated 40° C./75% RH)
Compound I 0.5 mg capsules; Accelerated 40° C./75% RH

| | Acceptance | | | | |
|---|---|---|---|---|---|
| Test | criteria | T0 | T1 | T3 | T6 |
| Assay | 90.0-110.0% | 97% | 96% | 95% | 95% |
| | of label claim | | | | |
| Related substances | | | | | |
| Reporting threshold | 0.1% | 0.2% (RRT0.42) | 0.4% (RRT0.42) | 0.4% (RRT0.42) | 0.6% (RRT0.42) |
| Unspecified impurity | NMT 1.0% | 0.2% (Total) | 0.4% (Total) | 0.4% (Total) | 0.05% (RRT0.74) |
| & deg. product | | | | | |
| Total impurities & | NMT 3.0% | | | | 0.7% (Total) |
| deg. Products | | | | | |

TABLE 14
Stability data for 0.5 mg Compound I GMP Clinical Capsules (Intermediate 30° C./65% RH)
Compound I 0.5 mg capsules; Intermediate 30° C./65% RH

| | Acceptance | | | | | | | |
|---|---|---|---|---|---|---|---|---|
| Test | criteria | T0 | T1 | T3 | T6 | T9 | T12 | T18 |
| Assay | 90.0-110.0% | 97% | 96% | 95% | 97% | 97% | 96% | 95% |
| | of label claim | | | | | | | |
| Related | | | | | | | | |
| substances | | | | | | | | |
| Reporting | 0.1% | | | | | | | |
| threshold | | | | | | | | |
| Unspecified | NMT 1.0% | 0.2% | 0.3% | 0.3% | 0.3% | 0.4% | 0.4% | 0.6% |
| impurity & | | (RRT0.42) | (RRT 0.42) | (RRT 0.42) | (RRT 0.42) | (RRT 0.43) | (RRT 0.42) | (RRT0.42) |
| deg. product | | | | | | | | |
| Total | NMT 3.0% | 0.2% | 0.3% | 0.3% | 0.3% | 0.4% | 0.4% | 0.6% |
| impurities | | (Total) | (Total) | (Total) | (Total) | (Total) | (Total) | (Total) |
| & deg. | | | | | | | | |
| Products | | | | | | | | |

TABLE 15
Stability data for 0.5 mg Compound I GMP Clinical Capsules (Long term 25° C./60% RH)
Compound I 0.5 mg capsules; Long term 25° C./60% RH

| | Acceptance | | | | | | | |
|---|---|---|---|---|---|---|---|---|
| Test | criteria | T0 | T1 | T3 | T6 | T9 | T12 | T18 |
| Assay | 90.0-110.0% | 97% | 95% | 97% | 97% | 98% | 98% | 95% |
| | of label claim | | | | | | | |
| Related | | | | | | | | |
| substances | | | | | | | | |
| Reporting | 0.1% | | | | | | | |
| threshold | | | | | | | | |
| Unspecified | NMT 1.0% | 0.2% | 0.3% | 0.3% | 0.3% | 0.4% | 0.3% | 0.5% |
| impurity & | | (RRT0.42) | (RRT0.42) | (RRT0.42) | (RRT0.42) | (RRT0.43) | (RRT0.42) | (RRT0.42) |
| deg. product | | | | | | | | |
| Total | NMT 3.0% | 0.2% | 0.3% | 0.3% | 0.3% | 0.4% | 0.3% | 0.5% |
| impurities | | (Total) | (Total) | (Total) | (Total) | (Total) | (Total) | (Total) |
| & deg. | | | | | | | | |
| Products | | | | | | | | |

TABLE 16
Stability data for 1.0 mg Compound I GMP Clinical Capsules (Accelerated 40° C./75% RH)
Compound I 1.0 mg capsules; Accelerated 40° C./75% RH

| | Acceptance | | | | |
|---|---|---|---|---|---|
| Test | criteria | T0 | T1 | T3 | T6 |
| Assay | 90.0-110.0% | 102% | 100% | 99% | 100% |
| | of label claim | | | | |
| Related substances | | | | | |
| Reporting threshold | 0.1% | 0.2% (RRT0.42) | 0.4% (RRT0.42) | 0.6% (RRT 0.42) | 0.6% (RRT 0.42) |
| Unspecified impurity | NMT 1.0% | 0.2% (Total) | 0.4% (Total) | 0.1% (RRT 1.20) | 0.1% (RRT 0.74) |
| & deg. product | | | | | |
| Total impurities & | NMT 3.0% | | | 0.7% (Total) | 0.7% (Total) |
| deg. Products | | | | | |

TABLE 17
Stability data for 1.0 mg Compound I GMP Clinical Capsules (Intermediate 30° C./65% RH)
Compound I 1.0 mg capsules; Intermediate 30° C./65% RH

| | Acceptance | | | | | | | |
|---|---|---|---|---|---|---|---|---|
| Test | criteria | T0 | T1 | T3 | T6 | T9 | T12 | T18 |
| Assay | 90.0-110.0% | 102% | 100% | 102% | 101% | 102% | 102% | 100% |
| | of label claim | | | | | | | |
| Related | | | | | | | | |
| substances | | | | | | | | |
| Reporting | 0.1% | | | | | | | |
| threshold | | | | | | | | |
| Unspecified | NMT 1.0% | 0.2% | 0.3% | 0.3% | 0.3% | 0.4% | 0.5% | 0.6% |
| impurity & | | (RRT0.42) | (RRT 0.42) | (RRT 0.42) | (RRT 0.42) | (RRT 0.42) | (RRT 0.42) | (RRT 0.42) |
| deg. product | | | | | | | | |
| Total | NMT 3.0% | 0.2% | 0.3% | 0.3% | 0.3% | 0.4% | 0.5% | 0.6% |
| impurities | | (Total) | (Total) | (Total) | (Total) | (Total) | (Total) | (Total) |
| & deg. | | | | | | | | |
| Products | | | | | | | | |

TABLE 18
Stability data for 1.0 mg Compound I GMP Clinical Capsules (Long term 25° C./60% RH)
Compound I 1.0 mg capsules; Long term 25° C./60% RH

| | Acceptance | | | | | | | |
|---|---|---|---|---|---|---|---|---|
| Test | criteria | T0 | T1 | T3 | T6 | T9 | T12 | T18 |
| Assay | 90.0-110.0% | 102% | 102% | 101% | 100% | 101% | 102% | 101% |
| | of label claim | | | | | | | |
| Related | | | | | | | | |
| substances | | | | | | | | |
| Reporting | 0.1% | | | | | | | 0.5% |
| threshold | | | | | | | | (RRT 0.42) |
| Unspecified | NMT 1.0% | 0.2% | 0.3% | 0.2% | 0.3% | 0.4% | 0.4% | 0.1% |
| impurity & | | (RRT0.42) | (RRT 0.42) | (RRT 0.42) | (RRT 0.42) | (RRT 0.43) | (RRT 0.42) | (RRT 0.75) |
| deg. product | | | | | | | | |
| Total | NMT 3.0% | 0.2% | 0.3% | 0.2% | 0.3% | 0.4% | 0.4% | 0.6% |
| impurities | | (Total) | (Total) | (Total) | (Total) | (Total) | (Total) | (Total) |
| & deg. | | | | | | | | |
| Products | | | | | | | | |

### Example 4

Table 19 below shows chemical instability (increase in related substances over time) for PEG1500+/−anti-oxidant (AO) and Gelucire 44/14 compared with good stability for TPGS+AO

TABLE 19
Chemical instability

| | | Storage | | | | | | | | | | | | | |
|---|---|---|---|---|---|---|---|---|---|---|---|---|---|---|---|
| | Vial | Temperature/ | RRT | | | | | | | | | | | | |
| Sample | no | Timepoint | 0.51 | 0.72 | 0.74 | 0.8 | 0.89 | 0.92 | 0.94 | 0.95 | 0.97 | 0.97 | 0.99 | 1.2 | Total |
| PEG1500 | A1 | Initial | 0.11 | | | | | | | | | | 0.08 | 0.06 | 0.25 |
| no anti- | A3 | 25° C./7 days | 0.11 | | | | | | | | | | 0.09 | 0.06 | 0.26 |
| oxidant | A5 | 25° C./14 days | | | | | | | | | | | 0.09 | 0.05 | 0.14 |
| | A9 | 40° C./7 days | 0.18 | | | | | | | | | | 0.22 | 0.06 | 0.46 |
| | A11 | 40° C./14 days | 0.24 | | 0.07 | | | 0.11 | 0.06 | 0.06 | 0.06 | | 0.31 | 0.06 | 0.97 |
| PEG1500 | D1 | Initial | 0.1 | | | | | | | | | | | 0.06 | 0.16 |
| & anti- | D3 | 25° C./7 days | 0.14 | | | | | | | | | | | 0.06 | 0.2 |
| oxidant | D5 | 25° C./14 days | 0.15 | | | | | | | | | | | 0.05 | 0.2 |
| | D9 | 40° C./7 days | 0.18 | | | | | | | | | | | 0.06 | 0.24 |
| | D11 | 40° C./14 days | 0.23 | | | | | | | | | | | 0.06 | 0.29 |
| Gelucire | B1 | Initial | | | | | | | | 0.07 | | | | 0.06 | 0.13 |
| 44/14 | B3 | 25° C./7 days | 0.13 | | | | | | | 0.11 | | | | 0.06 | 0.3 |
| | B5 | 25° C./14 days | 0.22 | | 0.05 | | | | | 0.12 | | | | 0.06 | 0.45 |
| | B9 | 40° C./7 days | 0.13 | | 0.07 | 0.07 | 0.07 | 0.09 | | 0.4 | | | | 0.06 | 0.89 |
| | B11 | 40° C./14 days | 0.18 | 0.08 | 0.17 | 0.16 | 0.14 | 0.17 | | 0.55 | 0.06 | 0.05 | 0.09 | 0.05 | 1.7 |
| TPGS & | C1 | Initial | | | | | | | | | | | | 0.08 | 0.08 |
| anti- | C3 | 25° C./7 days | | | | | | | | | | | | 0.08 | 0.08 |
| oxidant | C5 | 25° C./14 days | | | | | | | | | | | | | 0 |
| | C9 | 40° C./7 days | | | | | | | | | | | | 0.09 | 0.09 |
| | C11 | 40° C./14 days | | | | | | | | | | | | | 0 |

1. A semi-solid pharmaceutical formulation comprising:
Compound I; 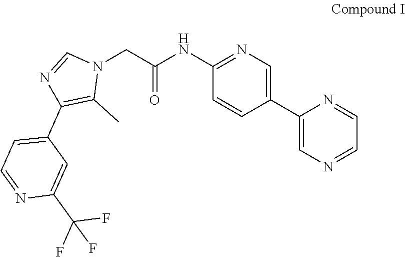 [SMILES: Cc1c(-c2ccnc(C(F)(F)F)c2)ncn1CC(=O)Nc1ccc(-c2cnccn2)cn1]
distributed in a semi-solid diluent.

2. The semi-solid pharmaceutical formulation of claim 1, wherein the semi-solid diluent is selected from the group comprising polyethylene glycol, a water soluble surfactant, a water dispersible surfactant, a hard fat, Vitamin E polyethylene glycol succinate, a polyoxyl 35 castor oil, a polyoxyl 40 castor oil, a glyceryl stearate, a glyceryl caprate/caprylate ester and medium chain triglycerides.

3. The semi-solid pharmaceutical formulation of claim 1 or claim 2, wherein the semi-solid diluent is selected from the group comprising PEG, PEG1000, Gelucire® 48/16, Gelucire® 44/14, Gelucire® 50/13, Gelucire® 43/01, Vitamin E polyethylene glycol succinate, polyoxyl 35 castor oil e.g. Kolliphor EL, polyoxyl 40 castor oil e.g. Kolliphor RH40, Solutol HS15 (polyoxyethylene esters of 12-monohydroxy stearic acid), glyceryl stearate (Imwitor 900), glyceryl caprate/caprylate ester e.g. Capmul MCM.

4. The semi-solid pharmaceutical formulation of any preceding claim, wherein the diluent is Vitamin E polyethylene glycol succinate.

5. The semi-solid pharmaceutical formulation of any preceding claim, wherein Compound I is present in an amount of from about 0.1% w/w to about 10% w/w.

6. The semi-solid pharmaceutical formulation of preceding claim, wherein Compound I is present in an amount of from about 0.5% w/w to about 5% w/w.

7. The semi-solid pharmaceutical formulation of preceding claim, wherein Compound I is present in an amount of from about 0.5% w/w to about 3% w/w.

8. The semi-solid pharmaceutical formulation of any preceding claim, wherein Compound I is present in an amount of from about 0.05 mg to about 5 mg.

9. The semi-solid pharmaceutical formulation of any preceding claim, wherein Compound I is present in an amount of from about 0.25 mg to about 2.5 mg.

10. The semi-solid pharmaceutical formulation of any preceding claim, wherein Compound I is present in an amount of from about 0.5 mg to about 1 mg.

11. The semi-solid pharmaceutical formulation of any preceding claim, wherein the formulation comprises:
Compound I present in an amount of from 0.5% w/w to 3% w/w; and
Vitamin E polyethylene glycol succinate in an amount of from 97% w/w to 99.5% w/w.

12. The semi-solid pharmaceutical formulation of any preceding claim, wherein the semi-solid pharmaceutical formulation is encapsulated in a gelatin capsule.

13. A method of making a semi-solid pharmaceutical formulation, the method comprising the steps of:
(i) melting a semi-solid diluent;
(ii) distributing Compound I in the molten semi-solid diluent to form a molten blend;
(iii) loading the molten blend into a capsule shell.

14. The method of claim 13, wherein the distributing comprises dissolving Compound I in the molten semi-solid diluent.

15. The method of claim 13 or claim 14, wherein the capsule is a gelatin capsule.

16. The method of claim 13 or claim 14, wherein step (i) is performed at a temperature of between about 45° C. and about 80° C.

17. The method of any of claims 13 to 16, wherein the semi-solid diluent is selected from the group comprising polyethylene glycol, a water soluble surfactant, a water dispersible surfactant, a hard fat, Vitamin E polyethylene glycol succinate, a polyoxyl 35 castor oil, a polyoxyl 40 castor oil, a glyceryl stearate, a glyceryl caprate/caprylate ester and medium chain triglycerides.

18. The method of any of claims 13 to 16, wherein the semi-solid diluent is selected from the group comprising PEG, PEG1000, Gelucire® 48/16, Gelucire® 44/14, Gelucire® 50/13, Gelucire® 43/01, Vitamin E polyethylene glycol succinate, polyoxyl 35 castor oil e.g. Kolliphor EL, polyoxyl 40 castor oil e.g. Kolliphor RH40, Solutol HS15 (polyoxyethylene esters of 12-monohydroxy stearic acid), glyceryl stearate (Imwitor 900), glyceryl caprate/caprylate ester e.g. Capmul MCM.

19. The method of any of claims 13 to 16, wherein the semi-solid diluent is selected from Vitamin E polyethylene glycol succinate and Gelucire® 44/14.

20. The method of any of claims 13 to 19, wherein Compound I is present in an amount of from 0.1% w/w to 10% w/w.

21. The method of claim 20, wherein Compound I is present in an amount of from 0.5% w/w to 3% w/w.

22. The method of claim 20, wherein Compound I is present in an amount of from 1% w/w to 2% w/w.

23. The method of any of claims 13 to 22, wherein Compound I is present in an amount of from about 0.05 mg to about 5 mg.

24. The method of claim 23, wherein Compound I is present in an amount of from about 0.25 mg to about 2.5 mg.

25. The method of any one of claims 13 to 15, wherein step (i) is performed at a temperature in the range from about 45° C. and about 80° C. and the diluent is Vitamin E polyethylene glycol succinate; and
Compound I is dissolved at an amount of from 0.5% w/w to 3% w/w.

26. The pharmaceutical formulation of any of claims 1 to 12 for use as a medicament.

27. The pharmaceutical formulation of any of claims 1 to 12 for use in the treatment of cancer.

28. The pharmaceutical formulation for use of claim 27, wherein the cancer is a solid tumour.

29. The pharmaceutical formulation for use of claim 27, wherein the cancer is selected from gastric cancer, thymus cancer, pancreatic cancer, and biliary cancer.
